(19) United States
(12) Patent Application Publication
(10) Pub. No.: US 2009/0099191 A1
(43) Pub. Date: Apr. 16, 2009

(54) INHIBITION OF NF-KB

(76) Inventor: Andrei V. Gudkov, East Aurora, NY (US)

Correspondence Address: POLSINELLI SHUGART PC, 700 W. 47TH STREET, SUITE 1000, KANSAS CITY, MO, 64112-1802, US

(21) Appl. No.: 12/278,124

(22) PCT Filed: Feb. 2, 2007
(86) PCT No.: PCT/IB07/04531

§ 371 Date: Nov. 4, 2008

Related U.S. Application Data
(60) Provisional application No. 60/743,221, filed on Feb. 2, 2006.

(51) Int. Cl. A61K 31/4985; A61K 31/473; A61K 31/4745; C12Q 1/02; A61P 35/04

(52) U.S. Cl. 514/250; 514/297; 514/292; 435/29

(57) ABSTRACT

The present invention is generally related to the modulation of cell growth or apoptosis. Compositions for modulating cell growth or apoptosis, methods of use thereof, and methods of identification thereof are described.

### BACKGROUND OF THE INVENTION

1. Field of the Invention

The present invention is generally related to the modulation of cell growth or apoptosis. More specifically, the present invention is related to compositions for modulating cell growth or apoptosis, methods of use thereof, and methods of identification thereof.

2. Description of Related Art

The frequency of cancer in humans has increased in the developed world as the population has aged. For some types of cancers and stages of disease at diagnosis, morbidity and mortality rates have not improved significantly in recent years in spite of extensive research. Induction of programmed cell death or apoptosis is one of the most attractive cancer treatment strategies.

p53 controls genetic stability and reduces the risk of cancer through induction of growth arrest or apoptosis in response to DNA damage or deregulation of proto-oncogenes. The efficacy of p53 as a tumor-preventing factor is reflected by the frequency of p53 loss in at least 50% of human tumors due to inactivating mutations. Several mechanisms of functional inactivation of wild type p53 have been described in human tumors, usually involving excessive degradation of p53 via proteasomes and mediated by Mdm2. Mdm2 is considered an attractive target for suppression by small molecules or other approaches in order to selectively kill tumor cells by restoring p53 function.

Renal cell carcinomas (RCC) maintain wild type but functionally inactive p53. The mechanism of p53 repression in RCC is dominant, which indicates the existence of a so far unknown molecular target for restoration of p53 function in cancer. There is a significant need to identify agents that are capable of restoring wild type p53 activity in tumor cells.

### SUMMARY OF THE INVENTION

A condition associated with NF-κB activity may be treated by administering to a patient in need thereof a composition comprising an inhibitor of NF-κB. The NF-κB activity may be constitutive, induced or at a basal level. The inhibition of NF-κB may activate p53. The inhibition of NF-κB may activate functionally silent p53. The condition treated may be cancer, inflammation, autoimmune disease, graft versus host disease, a condition associated with HIV infection, or pre-cancerous cells which have acquired dependence on constitutively active NF-κB. Forms of cancer, which may be treated, include, but are not limited to, renal cell carcinoma, sarcoma, prostate cancer, breast cancer, pancreatic cancer, myeloma, myeloid and lymphoblastic leukemia, neuroblastoma, glioblastoma or a cancer caused by HTLV infection.

The inhibitor of NF-κB may be a compound of the formula:

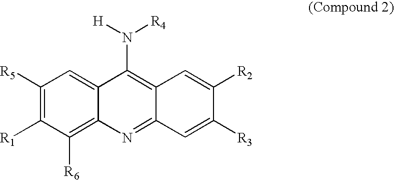

wherein,
R₁ is H or halogen;
R₂ is H or optionally substituted alkoxy group;
R₃ is H, optionally substituted alkoxy group or optionally substituted amino group;
R₄ is H, optionally substituted aliphatic group, optionally substituted aryl group, or optionally substituted heterocycle;
R₅ is H or optionally substituted alkoxy group;
R₆ is H or optionally substituted alkyl.

The inhibitor of NF-κB may also be a compound selected from the group consisting of:

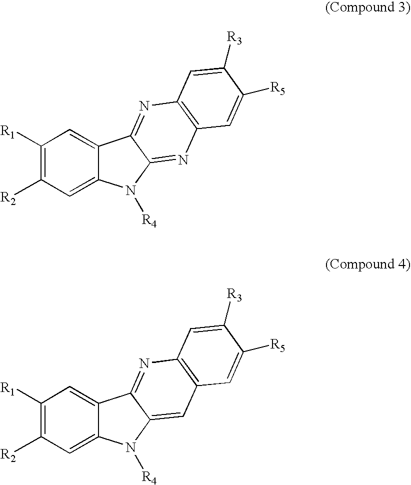

R₁-R₃ and R₅ are individually H or optionally substituted alkoxy; and
R₄ is H or optionally substituted aliphatic, aryl, or heterocycle.

The inhibitor of NF-κB may also be a compound set forth on FIGS. 2-5 and 7. The composition may further comprise an activator of a death receptor of a TNF family polypeptide. The activator may be a TNF polypeptide, such as NGF, CD40L, CD137L/4-1BBL, TNF-α, CD134L/OX40L, CD27L/CD70, FasL/CD95, CD30L, TNF-β/LT-α, LT-β, or TRAIL.

An agent that modulates functionally silent p53 may be identified by adding a candidate agent to a cell comprising a p53-responsive reporter and measuring the level of signal of the p53-responsive reporter. The agent may be identified by a difference in the signal compared to a control. The agent may increase or decrease the activity of p53. The cell may comprise a functionally silent p53.

An agent that modulates NF-κB may be identified by adding a candidate agent to a cell comprising a p53-responsive reporter and measuring the level of signal of the p53-responsive reporter. The agent may be identified by a difference in the signal compared to a control. The agent may increase or decrease the activity of NF-κB. The cell may comprise a functionally silent p53. The cell may also comprise an NF-κB transactivation complex.

### BRIEF DESCRIPTION OF THE DRAWINGS

FIG. 1 indicates that the restoration of p53-mediated transactivation in RCC cells is accompanied by death of RCC cells. FIG. 1A: p53-responsive reporter activity in RCC45ConALacZ cells transduced with different concentration of p53 or GFP expressing lentiviral vectors. β-galactosidase activity (ONPG staining) was measured 48 hours after lentiviral transduction and normalized by protein concentration. FIG. 1B: Cell survival was measured at 96 hours after lentiviral transduction by methylene blue staining and presented as a percentage of intensity of methylene blue staining of cell transduced with p53-virus to the same cells transduced with the same concentration of GFP-virus.

FIG. 2 shows primary hits from the initial screen that were used to synthesize focused libraries.

FIG. 3 indicates the p53 restoration activity of agents of the formula of compound 1. FIG. 3A: Choice of readout cells and setting of selection criterion. MCF7, ACHN, RCC26b and RCC45 cells all containing ConALacZ reporter were plated into 96 well plates and incubated in the medium containing different concentrations of doxorubicin for 24 hours. Then β-galactosidase activity was measured by ONPG staining and normalized by protein concentration. FIG. 3B indicates that 9AA causes the strongest activation of p53-dependent reporter in RCC45 cells. Agents of the formula of compound 1 were tested in dose dependent assay on p53 transactivation in RCC45ConALacZ cells. Bars represent relative activity of each compound calculated as a fold of p53 activation, induced by a compound, over the effect of 2 μM of doxorubicin (results of three experiments).

FIG. 4 shows compounds identified as p53 activators from the Class I focused library.

FIG. 5 shows compounds identified as p53 activators from the Class II focused library.

FIG. 6 shows a comparison of IC50% (concentration causing 50% decrease in number of cells, compared with untreated population) between quinacrine, and compound 662 (a primary hit from class 2). The assay was performed on 6 RCC cell lines, 6 non-RCC tumor cell lines and 2 normal cell strains (normal kidney epithelial cells and normal fibroblast). The dots on the graphs represent IC50 for each individual cell line from the indicated groups.

FIG. 7 shows compounds identified from screening compounds synthesized based on hits from the Class I and Class 2 focused libraries.

### DETAILED DESCRIPTION

Before the present compounds, products and compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

### 1. Definitions

The term “branched” as used herein refers to a group containing from 1 to 24 backbone atoms wherein the backbone chain of the group contains one or more subordinate branches from the main chain. Preferred branched groups herein contain from 1 to 12 backbone atoms. Examples of branched groups include, but are not limited to, isobutyl, t-butyl, isopropyl, —CH₂CH₂CH(CH3)CH₂CH₃, —CH₂CH(CH₂CH₃)CH₂CH₃, —CH₂CH₂C(CH₃)₂CH₃, —CH₂CH₂C(CH₃)₃ and the like.

The term “unbranched” as used herein refers to a group containing from 1 to 24 backbone atoms wherein the backbone chain of the group extends in a direct line. Preferred unbranched groups herein contain from 1 to 12 backbone atoms.

The term “cyclic” or “cyclo” as used herein alone or in combination refers to a group having one or more closed rings, whether unsaturated or saturated, possessing rings of from 3 to 12 backbone atoms, preferably 3 to 7 backbone atoms.

The term “lower” as used herein refers to a group with 1 to 6 backbone atoms.

The term “saturated” as used herein refers to a group where all available valence bonds of the backbone atoms are attached to other atoms. Representative examples of saturated groups include, but are not limited to, butyl, cyclohexyl, piperidine and the like.

The term “unsaturated” as used herein refers to a group where at least one available valence bond of two adjacent backbone atoms is not attached to other atoms. Representative examples of unsaturated groups include, but are not limited to, —CH₂CH₂CH═CH₂, phenyl, pyrrole and the like.

The term “aliphatic” as used herein refers to an unbranched, branched or cyclic hydrocarbon group, which may be substituted or unsubstituted, and which may be saturated or unsaturated, but which is not aromatic. The term aliphatic further includes aliphatic groups, which comprise oxygen, nitrogen, sulfur or phosphorous atoms replacing one or more carbons of the hydrocarbon backbone.

The term “aromatic” as used herein refers to an unsaturated cyclic hydrocarbon group having 4n+2 delocalized π(pi) electrons, which may be substituted or unsubstituted. The term aromatic further includes aromatic groups, which comprise a nitrogen atom replacing one or more carbons of the hydrocarbon backbone. Examples of aromatic groups include, but are not limited to, phenyl, naphthyl, thienyl, furanyl, pyridinyl, (is)oxazoyl and the like.

The term “substituted” as used herein refers to a group having one or more hydrogens or other atoms removed from a carbon or suitable heteroatom and replaced with a further group. Preferred substituted groups herein are substituted with one to five, most preferably one to three substituents. An atom with two substituents is denoted with “di,” whereas an atom with more than two substituents is denoted by “poly.” Representative examples of such substituents include, but are not limited to aliphatic groups, aromatic groups, alkyl, alkenyl, alkynyl, aryl, alkoxy, halo, aryloxy, carbonyl, acryl, cyano, amino, nitro, phosphate-containing groups, sulfur-containing groups, hydroxyl, alkylcarbonyloxy, arylcarbonyloxy, alkoxycarbonyloxy, aryloxycarbonyloxy, alkylcarbonyl, arylcarbonyl, alkoxycarbonyl, aminocarbonyl, alkylaminocarbonyl, dialkylaminocarbonyl, alkylthiocarbonyl, acylamino, amidino, imino, alkylthio, arylthio, thiocarboxylate, alkylsulfinyl, trifluoromethyl, azido, heterocyclyl, alkylaryl, heteroaryl, semicarbazido, thiosemicarbazido, maleimido, oximino, imidate, cycloalkyl, cycloalkylcarbonyl, dialkylamino, arylcycloalkyl, arylcarbonyl, arylalkylcarbonyl, arylcycloalkylcarbonyl, arylphosphinyl, arylalkylphosphinyl, arylcycloalkylphosphinyl, arylphosphonyl, arylalkylphosphonyl, arylcycloalkylphosphonyl, arylsulfonyl, arylalkylsulfonyl, arylcycloalkylsulfonyl, combinations thereof, and substitutions thereto.

The term “unsubstituted” as used herein refers to a group that does not have any further groups attached thereto or substituted therefor.

The term “alkyl” as used herein alone or in combination refers to a branched or unbranched, saturated aliphatic group. Representative examples of alkyl groups include, but are not limited to, methyl, ethyl, n-propyl, isopropyl, n-butyl, isobutyl, sec-butyl, tert-butyl, octyl, decyl, tetradecyl, hexadecyl, eicosyl, tetracosyl and the like.

The term “alkenyl” as used herein alone or in combination refers to a branched or unbranched, unsaturated aliphatic group containing at least one carbon-carbon double bond which may occur at any stable point along the chain. Representative examples of alkenyl groups include, but are not limited to, ethenyl, E- and Z-pentenyl, decenyl and the like.

The term “alkynyl” as used herein alone or in combination refers to a branched or unbranched, unsaturated aliphatic group containing at least one carbon-carbon triple bond which may occur at any stable point along the chain. Representative examples of alkynyl groups include, but are not limited to, ethynyl, propynyl, propargyl, butynyl, hexynyl, decynyl and the like.

The term “aryl” as used herein alone or in combination refers to a substituted or unsubstituted aromatic group, which may be optionally fused to other aromatic or non-aromatic cyclic groups. Representative examples of aryl groups include, but are not limited to, phenyl, benzyl, naphthyl, benzylidine, xylyl, styrene, styryl, phenethyl, phenylene, benzenetriyl and the like.

The term “alkoxy” as used herein alone or in combination refers to an alkyl, alkenyl or alkynyl group bound through a single terminal ether linkage. Examples of alkoxy groups include, but are not limited to, methoxy, ethoxy, n-propoxy, iso-propoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, 3-methylpentoxy, fluoromethoxy, difluoromethoxy, trifluoromethoxy, chloromethoxy, dichloromethoxy, and trichloromethoxy.

The term “aryloxy” as used herein alone or in combination refers to an aryl group bound through a single terminal ether linkage.

The term “halogen,” “halide” or “halo” as used herein alone or in combination refers to fluorine “F”, chlorine “Cl”, bromine “Br”, iodine “I”, and astatine “At”. Representative examples of halo groups include, but are not limited to, chloroacetamido, bromoacetamido, idoacetamido and the like.

The term “hetero” as used herein combination refers to a group that includes one or more atoms of any element other than carbon or hydrogen. Representative examples of hetero groups include, but are not limited to, those groups that contain heteroatoms including, but not limited to, nitrogen, oxygen, sulfur and phosphorus.

The term “heterocycle” as used herein refers to a cyclic group containing a heteroatom. Representative examples of heterocycles include, but are not limited to, pyridine, piperadine, pyrimidine, pyridazine, piperazine, pyrrole, pyrrolidinone, pyrrolidine, morpholine, thiomorpholine, indole, isoindole, imidazole, triazole, tetrazole, furan, benzofuran, dibenzofuran, thiophene, thiazole, benzothiazole, benzoxazole, benzothiophene, quinoline, isoquinoline, azapine, naphthopyran, furanobenzopyranone and the like.

The term “carbonyl” or “carboxy” as used herein alone or in combination refers to a group that contains a carbon-oxygen double bond. Representative examples of groups which contain a carbonyl include, but are not limited to, aldehydes (i.e., formyls), ketones (i.e., acyls), carboxylic acids (i.e., carboxyls), amides (i.e., amidos), imides (i.e., imidos), esters, anhydrides and the like.

The term “acryl” as used herein alone or in combination refers to a group represented by CH₂═C(Q)C(O)O— where Q is an aliphatic or aromatic group.

The term “cyano,” “cyanate,” or “cyanide” as used herein alone or in combination refers to a carbon-nitrogren double bond. Representative examples of cyano groups include, but are not limited to, isocyanate, isothiocyanate and the like.

The term “amino” as used herein alone or in combination refers to a group containing a backbone nitrogen atom. Representative examples of amino groups include, but are not limited to, alkylamino, dialkylamino, arylamino, diarylamino, alkylarylamino, alkylcarbonylamino, arylcarbonylamino, carbamoyl, ureido and the like.

The term “phosphate-containing group” as used herein refers to a group containing at least one phosphorous atom in an oxidized state. Representative examples include, but are not limited to, phosphonic acids, phosphinic acids, phosphate esters, phosphinidenes, phosphinos, phosphinyls, phosphinylidenes, phosphos, phosphonos, phosphoranyls, phosphoranylidenes, phosphorosos and the like.

The term “sulfur-containing group” as used herein refers to a group containing a sulfur atom. Representative examples include, but are not limited to, sulfhydryls, sulfenos, sulfinos, sulfinyls, sulfos, sulfonyls, thios, thioxos and the like.

The term “optional” or “optionally” as used herein means that the subsequently described event or circumstance may or may not occur, and that the description includes instances where said event or circumstance occurs and instances where it does not. For example, the phrase “optionally substituted alkyl” means that the alkyl group may or may not be substituted and that the description includes both unsubstituted alkyl and alkyl where there is a substitution.

The term “effective amount,” when used in reference to a compound, product, or composition as provided herein, means a sufficient amount of the compound, product or composition to provide the desired result. The exact amount required will vary depending on the particular compound, product or composition used, its mode of administration and the like. Thus, it is not always possible to specify an exact “effective amount.” However, an appropriate effective amount may be determined by one of ordinary skill in the art informed by the instant disclosure using only routine experimentation.

The term “suitable” as used herein refers to a group that is compatible with the compounds, products, or compositions as provided herein for the stated purpose. Suitability for the stated purpose may be determined by one of ordinary skill in the art using only routine experimentation.

As used herein, the terms “administer” when used to describe the dosage of a compound, means a single dose or multiple doses of the compound.

As used herein, “apoptosis” refers to a form of cell death that includes progressive contraction of cell volume with the preservation of the integrity of cytoplasmic organelles; condensation of chromatin (i.e., nuclear condensation), as viewed by light or electron microscopy; and/or DNA cleavage into nucleosome-sized fragments, as determined by centrifuged sedimentation assays. Cell death occurs when the membrane integrity of the cell is lost (e.g., membrane blebbing) with engulfment of intact cell fragments (“apoptotic bodies”) by phagocytic cells.

As used herein, the term “cancer” means any condition characterized by resistance to apoptotic stimuli.

As used herein, the term “cancer treatment” means any treatment for cancer known in the art including, but not limited to, chemotherapy and radiation therapy.

As used herein, the term “combination with” when used to describe administration of an aminoacridine and an additional treatment means that the aminoacridine may be administered prior to, together with, or after the additional treatment, or a combination thereof.

As used herein, the term “treat” or “treating” when referring to protection of a mammal from a condition, means preventing, suppressing, repressing, or eliminating the condition. Preventing the condition involves treating the mammal prior to onset of the condition. Suppressing the condition involves treating the mammal after induction of the condition but before its clinical appearance. Repressing the condition involves treating the mammal after clinical appearance of the condition such that the condition is reduced or maintained. Elimination the condition involves treating the mammal after clinical appearance of the condition such that the mammal no longer suffers the condition.

As used herein, the term “tumor cell” means any cell characterized by resistance to apoptotic stimuli.

### 2. NF-kB-Mediated Mechanism of p53 Suppression in Tumors

The present invention is related to the discovery that p53 may be activated in those cancer cells that have functionally blocked p53 by inhibiting NF-κB activity. Inactivation of p53 pathway in tumors is a much broader phenomenon than p53 mutations. Even if a tumor maintains wild type p53, its function is usually either completely or partially lost. These cases are especially interesting from the therapeutic standpoint since p53 in such cancers can be viewed as a target for a pharmacological reactivation. There are some types of tumors in which p53 activity is blocked by tissue-specific mechanisms. Thus, Hdm2 overexpression is especially frequent in sarcomas, while E6 of human papilloma virus inactivates p53 in the majority of cervical carcinomas. RCC provides another example of that kind of tumor, which is especially interesting for the analysis since wild type p53 in RCC, as we recently reported, is repressed by an unknown dominant mechanism that is likely to be tissue specific. Hence, p53 reactivation seems to be an attractive strategy for treatment of this, so far, incurable form of cancer as well as other cancers with similar mechanisms for inactivating p53.

NF-κB activity is linked with the suppression of apoptosis in vitro and in vivo. Consistently, many apoptosis-resistant tumors acquire constitutive activation of NF-κB. Activation of NF-κB in tumor cells presumably contributes to their malignant phenotype by providing resistance to both natural (e.g., TNF, Fas or TRAIL) and pharmacological (chemotherapeutic drugs) death stimuli. While constitutively active NF-κB has been described in many tumor types, the connection between activation of NFκB and the inhibition of p53 has failed to be fully appreciated.

Cancers, such as those with functional or wild-type p53, may be treated by inhibiting NF-κB activity, which may lead to restoration of wild-type p53 activity and its activation. Inhibitors of NF-κB activity may also be used to sensitize cancers to p53-dependent and p53-independent apoptosis by treatments such as chemotherapeutics, radiotherapy or natural death ligands, such as TNF polypeptides. Regardless of their p53 status, the majority of human cancers have constitutively hyperactivated NF-κB. As a results, inhibitors of NF-κB may be used for treatment of any tumor regardless of their p53 status due to the reprogramming of transactivation NF-κB complexes into transrepression complexes.

### 3. NF-κB Inhibiting Agent

a. Aminoacridines

Aminoacridines are representative examples of agents which may be used to inhibit NF-κB activity. The aminoacridine may be of the following formula:

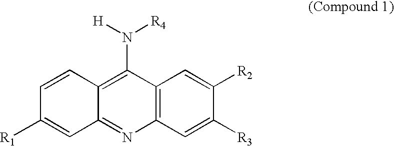

wherein,
R₁ is H or halogen;
R₂ is H or optionally substituted alkoxy;
R₃ is H or optionally substituted alkoxy; and
R₄ is H or optionally substituted aliphatic, aryl, or heterocycle.

The aminoacridine may also be of the following formula:

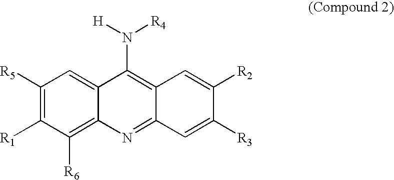

wherein,
R₁ is H or halogen;
R₂ is H or optionally substituted alkoxy group;
R₃ is H, optionally substituted alkoxy group or optionally substituted amino group;
R₄ is H, optionally substituted aliphatic group, optionally substituted aryl group, or optionally substituted heterocycle;
R₅ is H or optionally substituted alkoxy group;
R₆ is H or optionally substituted alkyl.
   The R₄ of Compound 2 may be 2- or 5- optionally substituted 4-ethylidene-2,4-dihidro-3-H-pyrazol-3-one.

Representative examples of aminoacridines include, but are not limited to, 9-aminoacridine or Mepacrine, which is otherwise known as Quinacrine, as well as those aminoacridines described in the Examples 2-5. The use of aminoacridines to sensitize tumor cells is attractive because many aminoacridines have limited side effects.

9AA has been used as therapeutic agent since 1942. Certain 9AA derivatives have been believed to be intercalating capable of DNA damaging activity; however, we found that 9AA and quinacrine did not show DNA damaging activity. Both 9aa and quinacrine were found to be more toxic to tumor than to normal cells in vitro and in vivo. Moreover, both compounds were shown to be capable of p53 activation and p53-dependent killing of a variety of tumor cell types, besides RCC. p53 dependence of their anti-tumor activity clearly distinguishes the aminoacridines from conventional chemotherapeutic drugs based on their targeting of tumors with wild type or functional p53.

Aminoacridines do not fit any known category of p53 activating agents. Although they may cause accumulation of p53, they do not induce p53 phosphorylation, unlike DNA damaging drugs. Moreover, aminoacridines do not cause DNA damage. Instead, the primary effect of aminoacridines appeared to be not p53 activation but repression of NF-κB, which later leads to p53 induction. Importantly, inhibition of NF-κB activates p53 function in a cell in which it cannot be “waked up” by any of the direct approaches to p53 activation, including introduction of Arf, knockdown of Hdm2 or ectopic overexpression of p53.

Inhibition of NF-κB is usually achieved through stabilization of the main negative regulator of NF-κB, IκB. Genetically, it can be done by mutating regulatory phosphorylation sites of this protein and pharmacologically—through inhibition of upstream kinases leading to a block of IκB phosphorylation. Many known chemical inhibitors of NF-κB act through this mechanisms. Stabilization of IκB results in cytoplasmic sequestration and functional inactivation of NF-κB complexes as transcription factors.

The activity of aminoacridines may be superior to previous drugs since they promote strong accumulation of NF-κB complexes in the nuclei in response to activating stimuli accompanied with a complete repression of transactivation. Hence, aminoacridines may inhibit NF-κB by a mechanism acting downstream of IκB and involving conversion of NF-κB into an inactive complex. The lack of NF-κB-dependent transcription may lead to the depletion of the pool of IκB (that is a direct transcription target of NF-κB) and retention of NF-κB in the nucleus due to the lack of nuclear export, normally exerted by IκB. Interestingly, the knockout of any of the cellular factors involved in NF-κB activation (IKKα, IKKβ, TBK1, PKC-zeta) does not imitate the effect of aminoacridines, suggesting that none of them is a target of aminoacridines. It has recently demonstrated that nuclear accumulation of inactive NF-κB complexes, containing p65, occurs after cell treatment with UV, doxorubicin and daunorobicin; however, none of these treatments is comparable with aminoacridines in activating p53, presumably due to weaker NF-κB inhibitory activity.

The aminoacridines may be effective not only against the IκB phosphorylation arm of NF-κB signaling (“canonical” NF-κB activation pathway), but also through alternative mechanisms of NF-κB activation. This is supported by the ability of aminoacridines, such as 9AA, to block stimulated NF-κB activity and also effectively reduce basal levels of constitutive NF-κB activity in tumor cells. By contrast, IKK2 inhibitors are only able to block stimulated NF-κB activity.

b. Ellipticines

The agent used to inhibit NF-κB activity may also be an ellepticine-like compound. The ellepticine-like compound may be of one of the following formulas:

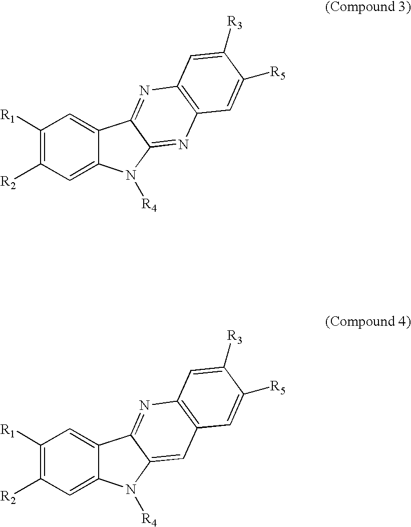

R₁-R₃ and R₅ are individually H or optionally substituted alkoxy; and
R₄ is H or optionally substituted aliphatic, aryl, or heterocycle.
   The ellepticine-like agent may also be a compound described in Examples 2-5. The ellepticine-like agent may be used to inhibit NF-κB activity.

The natural plant product ellipticine was isolated in 1959 from the Australian evergreen tree of the Apocynaceae family. The compound was initially an extremely promising anticancer drug. The planar polycyclic structure was found to interact with DNA through intercalation, exhibiting a high DNA binding affinity (10⁶ M⁻¹). The presence of protonatable ring nitrogens distinguished ellipticine from other simple intercalators. Both monocationic and uncharged species were found to be present under physiological conditions. The positive charge stabilized the binding of ellipticine to nucleic acids, while the more lipophilic uncharged compound was shown to readily penetrate membrane barriers. The structural nature of these compounds provides a basis for multiple modes of action, including DNA binding, interactions with membrane barriers, oxidative bioactivation and modification of enzyme function; most notably that of topoisomerase II and telomerase. Pharmacologically, a number of toxic side effects have been shown to be problematic. We have made structural medications to ellipticine based on rational drug design. A number of successful ellipticine analogs have been designed and synthesized with improved toxicities and anticancer activities.

### 4. Compositions

The present invention relates to a composition comprising an agent and optionally a chemotherapeutic. The present invention also relates to a composition comprising an agent and optionally a TNF polypeptide.

a. Chemotherapeutic

The chemotherapeutic may be any pharmacological agent or compound that induces apoptosis. The pharmacological agent or compound may be, for example, a small orgnanic molecule, peptide, polypeptide, nucleic acid, or antibody.

The chemotherapeutic may be a cytotoxic agent or cytostatic agent, or combination thereof. Cytotoxic agents prevent cancer cells from multiplying by: (1) interfering with the cell's ability to replicate DNA and (2) inducing cell death and/or apoptosis in the cancer cells. Cytostatic agents act via modulating, interfering or inhibiting the processes of cellular signal transduction which regulate cell proliferation and sometimes at low continuous levels.

Classes of compounds that may be used as cytotoxic agents include the following: alkylating agents (including, without limitation, nitrogen mustards, ethylenimine derivatives, alkyl sulfonates, nitrosoureas and triazenes): uracil mustard, chlormethine, cyclophosphamide (Cytoxan®), ifosfamide, melphalan, chlorambucil, pipobroman, triethylene-melamine, triethylenethiophosphoramine, busulfan, carmustine, lomustine, streptozocin, dacarbazine, and temozolomide; antimetabolites (including, without limitation, folic acid antagonists, pyrimidine analogs, purine analogs and adenosine deaminase inhibitors): methotrexate, 5-fluorouracil, floxuridine, cytarabine, 6-mercaptopurine, 6-thioguanine, fludarabine phosphate, pentostatine, and gemcitabine; natural products and their derivatives (for example, vinca alkaloids, antitumor antibiotics, enzymes, lymphokines and epipodophyllotoxins): vinblastine, vincristine, vindesine, bleomycin, dactinomycin, daunorubicin, doxorubicin, epirubicin, idarubicin, ara-c, paclitaxel (paclitaxel is commercially available as Taxol®), mithramycin, deoxyco-formycin, mitomycin-c, 1-asparaginase, interferons (preferably IFN-α), etoposide, and teniposide. Other proliferative cytotoxic agents are navelbene, CPT-11, anastrazole, letrazole, capecitabine, reloxafine, cyclophosphamide, ifosamide, and droloxafine.

Microtubule affecting agents interfere with cellular mitosis and are well known in the art for their cytotoxic activity. Microtubule affecting agents useful in the invention include, but are not limited to, allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), Taxol® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, and discodermolide (see Service, (1996) Science, 274:2009) estramustine, nocodazole, MAP4, and the like. Examples of such agents are also described in Bulinski (1997) J. Cell Sci. 110:3055 3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; and Panda (1996) J. Biol. Chem. 271:29807-29812.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Cytostatic agents that may be used include, but are not limited to, hormones and steroids (including synthetic analogs): 17.alpha.-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, hlorotrianisene, hydroxyprogesterone, aminoglutethimide, estramustine, medroxyprogesteroneacetate, leuprolide, flutamide, toremifene, zoladex.

Other cytostatic agents are antiangiogenics such as matrix metalloproteinase inhibitors, and other VEGF inhibitors, such as anti-VEGF antibodies and small molecules such as ZD6474 and SU6668 are also included. Anti-Her2 antibodies from Genetech may also be utilized. A suitable EGFR inhibitor is EKB-569 (an irreversible inhibitor). Also included are Imclone antibody C225 immunospecific for the EGFR, and src inhibitors.

Also suitable for use as an cytostatic agent is Casodex® (bicalutamide, Astra Zeneca) which renders androgen-dependent carcinomas non-proliferative. Yet another example of a cytostatic agent is the antiestrogen Tamoxifen® which inhibits the proliferation or growth of estrogen dependent breast cancer. Inhibitors of the transduction of cellular proliferative signals are cytostatic agents. Representative examples include epidermal growth factor inhibitors, Her-2 inhibitors, MEK-1 kinase inhibitors, MAPK kinase inhibitors, PI3 inhibitors, Src kinase inhibitors, and PDGF inhibitors.

b. TNF Polypeptides

The TNF polypeptide may be a member of the TNF superfamily of ligands. Representative examples of TNF polypeptides include, but are not limited to, NGF, CD40L, CD137L/4-1BBL, TNF-α, CD134L/OX40L, CD27L/CD70, FasL/CD95, CD30L, TNF-β/LT-α, LT-β, and TRAIL. Members of the TNF superfamily are natural proteins that are implicated in the maintenance and function of the immune system and that can trigger apoptosis. The TNF polypeptide may be TRAIL, which induces apoptosis mainly in tumor but not in normal cells.

The activity of these so-called “death ligands” is believed to be mediated by binding with members of the TNF receptor family, which contain structurally similar death domains in their intracellular portions. Ligation of these receptors, specific for each death ligand, trigger activation of a cascade of events resulting in caspase activation. Representative examples of TNF-R receptors bound by the TNF polypeptides include, but are not limited to, LNGFR/p75, CD40, CD 137/4-1 BB/ILA, TNFRI/p55/CD 120a, TNFRII/p75/CD120b, CD 134/OX40/ACT35, CD27, Fas/CD95/APO-1, CD30/Ki-1, LT-β R, DR3, DR4, DR5, DcR1/TRID, TR2, GITR and osteoprotegerin.

Due to their unique ability to induce apoptosis in tumor cells, TNF family members are considered to be potential anticancer pharmaceuticals. However, many tumor cells escape pro-apoptotic action of death ligands, thereby reducing the use of these agents to death ligand-sensitive cancers and allowing the tumor to escape host immune response. The use of an inhibitor of NF-kB may be used to sensitize tumor cells to the killing of a death ligand, such as a TNF polypeptide.

It also contemplated that other agents may be used in the place of the TNF polypeptide. For example, an antibody may be used that mimics the activity of a TNF polypeptide. Representative examples of such antibodies include, but are not limited to, an agonist antibody to FAS, TRAIL receptor or TNFR. In addition, aptamers and other synthetic ligands capable to activate the corresponding receptors may be used.

c. Salts

The active agents of the compositions may be useful in various pharmaceutically acceptable salt forms. The term “pharmaceutically acceptable salt” refers to those salt forms which would be apparent to the pharmaceutical chemist, i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients or their pharmaceutically acceptable salts in combination with pharmaceutically acceptable carriers.

Pharmaceutically acceptable salts of the active agents include, but are not limited to, salts formed with a variety of organic and inorganic acids such as hydrogen chloride, hydroxymethane sulfonic acid, hydrogen bromide, methanesulfonic acid, sulfuric acid, acetic acid, trifluoroacetic acid, maleic acid, benzenesulfonic acid, toluenesulfonic acid, sulfamic acid, glycolic acid, stearic acid, lactic acid, malic acid, pamoic acid, sulfanilic acid, 2-acetoxybenzoic acid, fumaric acid, toluenesulfonic acid, methanesulfonic acid, ethanedisulfonic acid, oxalic acid, isethonic acid, and include various other pharmaceutically acceptable salts, such as, e.g., nitrates, phosphates, borates, tartrates, citrates, succinates, benzoates, ascorbates, salicylates, and the like. Cations such as quaternary ammonium ions are contemplated as pharmaceutically acceptable counterions for anionic moieties. In addition, pharmaceutically acceptable salts of the compounds of the present invention may be formed with alkali metals such as sodium, potassium and lithium; alkaline earth metals such as calcium and magnesium; organic bases such as dicyclohexylamine, tributylamine, and pyridine; and amino acids such as arginine, lysine and the like.

The pharmaceutically acceptable salts may be synthesized by conventional chemical methods. Generally, the salts are prepared by reacting the free base or acid with stoichiometric amounts or with an excess of the desired salt-forming inorganic or organic acid or base, in a suitable solvent or solvent combination.

In general, the counterions of the salts may be determined by the reactants used to synthesized the compounds. There may be a mixture of counterions of the salts, depending on the reactants. For example, where NaI is added to facilitate the reaction the counterion may be a mixture of Cl and I counter anions. Furthermore preparatory HPLC may cause the original counterion to be exchanged by acetate anions when acetic acid is present in the eluent. The counterions of the salts may be exchanged to a different counterion. The counterions are preferably exchanged for a pharmaceutically acceptable counterion to form the salts described above. Procedures for exchanging counterions are described in WO 2002/042265, WO 2002/042276 and S. D. Clas, “Quaternized Colestipol, an improved bile salt adsorbent: In Vitro studies.” Journal of Pharmaceutical Sciences, 80(2): 128-131 (1991), the contents of which are incorporated herein by reference. For clarity reasons, the counterions may not be explicitly shown in the chemical structures herein.

d. Formulations

The composition may further comprise one or more pharmaceutically acceptable additional ingredient(s) such as alum, stabilizers, antimicrobial agents, buffers, coloring agents, flavoring agents, adjuvants, and the like.

The composition may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

The composition may also be liquid formulations including, but not limited to, aqueous or oily suspensions, solutions, emulsions, syrups, and elixirs. The composition may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

The composition may also be formulated as suppositories, which may contain suppository bases including, but not limited to, cocoa butter or glycerides. The composition may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane. The composition may also be formulated transdermal formulations comprising aqueous or nonaqueous vehicles including, but not limited to, creams, ointments, lotions, pastes, medicated plaster, patch, or membrane.

The composition may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The composition may also be formulated as a depot preparation, which may be administered by implantation or by intramuscular injection. The composition may be formulated with suitable polymeric or hydrophobic materials (as an emulsion in an acceptable oil, for example), ion exchange resins, or as sparingly soluble derivatives (as a sparingly soluble salt, for example).

The composition may also be formulated as a liposome preparation. The liposome preparation can comprise liposomes which penetrate the cells of interest or the stratum corneum, and fuse with the cell membrane, resulting in delivery of the contents of the liposome into the cell. For example, liposomes may be used such as those described in U.S. Pat. No. 5,077,211, U.S. Pat. No. 4,621,023 or U.S. Pat. No. 4,508,703, which are incorporated herein by reference. A composition intended to target skin conditions can be administered before, during, or after exposure of the skin of the mammal to UV or agents causing oxidative damage. Other suitable formulations can employ niosomes. Niosomes are lipid vesicles similar to liposomes, with membranes consisting largely of non-ionic lipids, some forms of which are effective for transporting compounds across the stratum corneum.

### 5. Treatment

The composition may be used for treating a condition associated with NF-kB activity in vivo by administering to a patient in need thereof an agent. The NF-κB activity may be at any level, the reduction of which would lead to treatment of the condition. The NF-κB activity may also be at a basal level. The NF-κB activity may also be at a constitutive level. The NF-κB activity may also be at an induced constitutive level.

The condition associated with NF-kB activity may be cancer. A variety of cancers may be treated including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, renal, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, teratocarcinoma, renal cell carcinoma (RCC), pancreatic cancer, myeloma, myeloid and lymphoblastic leukemia, neuroblastoma, and glioblastoma.

Transformation induced by tax of HTLV, a causative agent of human adult T-lymphoblastic leukemia (ATL), may share the same molecular targets involved in RCC. For example, NF-kB is constitutively active in tax-transformed cells. Similar to RCC, p53 activity is inhibited through activation of NF-kB in tax-transformed cells and p53 inhibition does not involve sequestering of p300. Based on the shared mechanism of p53 inactivation, the compositions may also be used to treat HTLV-induced leukemia. Regardless of their p53 status, the majority of human cancers have constitutively hyperactivated NF-kB. The composition may also be capable of inhibiting NF-kB by reprogramming transactivation NF-kB complexes into transrepression complexes, which may also be used for treatment of any tumor regardless of their p53 status. The compositions may also be used for treating HIV infections since HIV LTRs are strongly dependent on NF-kB activity.

The composition may also be used as an adjuvant therapy to overcome anti-cancer drug resistance that may be caused by constitutive NF-kB activation. The anti-cancer drug may be a chemotherapeutic described herein.

a. Administration

The composition may be administered simultaneously or metronomically with other anti-cancer treatments such as chemotherapy and radiation therapy. The term “simultaneous” or “simultaneously” as used herein, means that the other anti-cancer treatment and the composition is administered within 48 hours, 24 hours, 12 hours, 6 hours, 3 hours or less, of each other. The term “metronomically” as used herein means the administration of the composition at times different from the chemotherapy and at certain frequency relative to repeat administration and/or the chemotherapy regiment.

The composition may be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular. The composition may also be administered in the form of an implant, which allows slow release of the composition as well as a slow controlled i.v. infusion.

b. Dosage

A therapeutically effective amount of an agent required for use in therapy varies with the nature of the condition being treated, the length of time that activity is desired, and the age and the condition of the patient, and is ultimately determined by the attendant physician. The desired dose may be conveniently administered in a single dose, or as multiple doses administered at appropriate intervals, for example as one, two, three, four or more subdoses per day. Multiple doses often are desired, or required.

When given in combination with other therapeutics, the composition may be given at relatively lower dosages. In addition, the use of targeting agents may allow the necessary dosage to be relatively low. Certain compositions may be administered at relatively high dosages due to factors including, but not limited to, low toxicity, high clearance, low rates of cleavage of the tertiary amine. As a result, the dosage of a composition may be from about 1 ng/kg to about 200 mg/kg, about 1 μg/kg to about 100 mg/kg, or about 1 mg/kg to about 50 mg/kg. The dosage of a composition may be at any dosage including, but not limited to, about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

### 6. Diagnostic Methods

The composition may also be used to diagnose whether a tumor of a patient is capable of being treated by the composition. A sample of the tumor may be obtained from the patient. Cells of the tumor may then be transduced with a p53 reporter system, such as a p53-responsive lacZ reporter. The transduced cells may then be incubated with the composition. The production of a p53-mediated signal above controls indicates that the tumor may be treated by the composition.

### 7. Screening Methods

The present invention also relates to methods of identifying agents that modulate NF-κB activity. An agent that modulates NF-κB activity may be identified by a method comprising adding a candidate modulator of NF-κB activity to a cell-based NF-κB activated expression system, whereby a modulator of NF-κB activity is identified by the ability to alter the level of NF-κB activated expression. An agent that modulates NF-κB activity may also be identified by a method comprising adding a candidate modulator of NF-κB activity to a cell-based p53 activated expression system, whereby a modulator of NF-κB activity is identified by the ability to alter the level of p53 activated expression. An agent that modulates NF-κB activity may also be identified by a method comprising adding an agent and a candidate modulator of NF-κB activity to an NF-κB or p53 activated expression system, comparing the level of NF-κB or p53 activated expression to a control, whereby a modulator of NF-κB activity is identified by the ability to alter the level of NF-κB or p53 activated expression system compared to the control.

The cell may comprise a functionally silent p53. The cell may also comprise an NF-κB transactivation complex. The p53 activated expression system may be in a renal carcinoma cell line. The cell line may also be a sarcoma cell line. The cell line may also be a cell line with amplified mdm2. The cell line may also be a cell line that expresses HPV-E6 or is capable thereof.

Candidate agents may be present within a library (i.e., a collection of compounds). Such agents may, for example, be encoded by DNA molecules within an expression library. Candidate agent be present in conditioned media or in cell extracts. Other such agents include compounds known in the art as “small molecules,” which have molecular weights less than 10⁵ daltons, preferably less than 10⁴ daltons and still more preferably less than 10³ daltons. Such candidate agents may be provided as members of a combinatorial library, which includes synthetic agents (e.g., peptides) prepared according to multiple predetermined chemical reactions. Those having ordinary skill in the art will appreciate that a diverse assortment of such libraries may be prepared according to established procedures, and members of a library of candidate agents can be simultaneously or sequentially screened as described herein.

The screening methods may be performed in a variety of formats, including in vitro, cell-based and in vivo assays. Any cells may be used with cell-based assays. Preferably, cells for use with the present invention include mammalian cells, more preferably human and non-human primate cells. Cell-base screening may be performed using genetically modified tumor cells expressing surrogate markers for activation of NF-κB and/or p53. Such markers include, but are not limited to, bacterial β-galactosidase, luciferase and enhanced green fluorescent protein (EGFP). The amount of expression of the surrogate marker may be measured using techniques standard in the art including, but not limited to, colorimetery, luminometery and fluorimetery. Representative examples of cells that may be used in cell-based assays include, but are not limited to, renal cell carcinoma cells.

The conditions under which a suspected modulator is added to a cell, such as by mixing, are conditions in which the cell can undergo apoptosis or signaling if essentially no other regulatory compounds are present that would interfere with apoptosis or signaling. Effective conditions include, but are not limited to, appropriate medium, temperature, pH and oxygen conditions that permit cell growth. An appropriate medium is typically a solid or liquid medium comprising growth factors and assimilable carbon, nitrogen and phosphate sources, as well as appropriate salts, minerals, metals and other nutrients, such as vitamins, and includes an effective medium in which the cell can be cultured such that the cell can exhibit apoptosis or signaling. For example, for a mammalian cell, the media may comprise Dulbecco's modified Eagle's medium containing 10% fetal calf serum.

Cells may be cultured in a variety of containers including, but not limited to tissue culture flasks, test tubes, microtiter dishes, and petri plates. Culturing is carried out at a temperature, pH and carbon dioxide content appropriate for the cell. Such culturing conditions are also within the skill in the art.

Methods for adding a suspected modulator to the cell include, but are not limited to, electroporation, microinjection, cellular expression (i.e., using an expression system including naked nucleic acid molecules, recombinant virus, retrovirus expression vectors and adenovirus expression), use of ion pairing agents and use of detergents for cell permeabilization.

The present invention has multiple aspects, illustrated by the following non-limiting examples.

### EXAMPLES

### Materials and Methods

### Cells

Renal cell carcinoma cell lines used, RCC45, RCC54 and ACHN are described in Gurova, et al. (2004). Cancer Res 64, 1951-1958. H1299, HT1080, MCF7, LNCaP, PC3, DU145, HCT116, SK-N-SH, W138 cells were obtained from ATCC. The primary culture of normal kidney epithelial cells (NKE) was provided by J. Didonato (Cleveland Clinic Foundation, OH). 041 fibroblast cell line from Li-Fraumeni patient was provided by G. Stark. Mel7 and Mel29 cells are melanoma cell lines, described in Kichina, et al. (2003). Oncogene 22, 4911-4917. All cells were maintained in RPMI 1640 medium, supplemented with 10% FBS, 1 mM sodium pyruvate, 10 mM Hepes buffer, 55 nM β-mercaptoethanol and antibiotics.

Reporter cell lines with p53 responsive β-galactosidase were described in Gurova, et al. (2004). Cancer Res 64, 1951-1958. Reporter cell lines with p53 responsive luciferase was generated by transfection of p21-ConALuc plasmid with following selection on G418. Reporter cell lines with NF-κB-dependent luciferase were obtained by cotransfection of pNF-κBLuc and pEGFP-mito (Clontech) plasmids followed by selection on G418 (marker provided by pEGFP-mito plasmid). Reporter cell lines with myc, or Clock/Bmal responsive reporters were kindly provided by C. Burkhart and M. Antoch (Cleveland Clinic Foundation, OH).

Cells with inhibited p53 expression were generated by retroviral transduction of pBabeH1-sip53 or pBabeH1-siGFP vectors for siRNA expression followed by selection on puromycin.

### Plasmids

p53, Arf expression vectors, pBabeH1-siHdm2, p21-ConALuc reporter plasmid are described in Gurova, et al. (2004). Cancer Res 64, 1951-1958. pNF-κBLuc plasmid was provided by N. Neznanov (Cleveland Clinic Foundation, ref. 59). pcDNA3 vector expressing pss-IκB was provided by I. Budunova (Northwestern University). pBabeH1-sip53 and pBabeH1-siGFP vectors for siRNA expression were generated by insertion of H1promoter and 64 oligonucleotide loop template for siRNA expression into left LTR of pBabeH1-puro vector analogously to pBabeH1-siHDM2 vector, described in Gurova, et al. (2004). Cancer Res 64, 1951-1958. Sequences for siRNA against p53 and GFP are described in Brummelkamp, et al. (2002). Science 296, 550-553. Lentiviral plasmids for p53 or GFP expression are described in Gurova, et al. (2004). Cancer Res 64, 1951-1958.

### Chemicals

DiverSet library of 34,000 chemical compounds was obtained from Chembridge, Inc. Focused libraries of around 30d9 and 9AA were provided by Chembridge, Inc. Other chemicals were obtained from Sigma.

### Chemical Library Screening

2×10⁴ of RCC45ConALacZ cells were plated into wells of 96 well plates in 200 μL of phenol-red free RPMI medium with standard additives. After overnight incubation library of chemical compounds in DMSO solution together with controls was added with the help of plastic bacterial replicators (200+/−100mL). Final concentration of compounds was around 5 μg/ml. Negative control was DMSO, positive control was doxorubicin solution (0.2, 0.6 and 2 μM). After 24 hours lysis buffer with ONPG was added directly to the medium on ice. After 3 hours of incubation at 37° C., β-galactosidase activity was estimated by reading absorbance values Wallack 1420 plate reader (Perkin Elmer) at λ=430 nm. All compounds, inducing ONPG reaction stronger than the most effective concentration of doxorubicin was considered as primary hits.

### Reporter Assays

For cotransfection set-up, 2×10⁵ cells were plated into 6 well plates and, after overnight incubation, transfected with Lipofectamin Plus reagent (Gibco BRL) with 0.5 μg of reporter plasmids (p21-ConALuc or pNF-κBLuc) in combination with different concentrations of p53, Arf, Ss-IκB, or siHDM2 expressing plasmids. Corresponding empty vectors were added into all transfections up to 2 μg of total DNA amount. Normalization of transfection efficiency was done by adding 0.2 μg of pCMV-LacZ plasmid. Luciferase activity and β-galactosidase activity was measured in lysates prepared 48 hours after transfection with Cell Lysis Buffer (Promega) by luciferase assay system (Promega) or β-galactosidase enzyme system (Promega). Luminometric and colorimetric reactions were read on the Wallack 1420 plate reader (Perkin Elmer). Integrated reporter set-up. 2×10⁴ of cells with integrated reporter were plated in 96 well plates. After overnights incubation chemical compounds or media from lentivirus producing cells were added. At different time points cell lysates were prepared using Reporter Lysis Buffer (Promega). Luciferase or β-galactosidase activity and protein concentration were measured in aliquots of cell lysates using standard kits (Promega, Luciferase and β-galactosidase assay systems, Biorad Protein Assay Kit).

### Cell Survival Assays

5×10³ of cells were plated in 6 well plates and treated with different concentrations of drugs for 24 hours. Then fresh drug-free medium was added. Number of colonies was estimated after 5-6 days of incubation. Cell survival was estimated as a percentage of intensity of methylene blue staining of treated cells, comparing with untreated control (methylene blue from stained colonies was extracted by 0.1% of SDS and quantitated spectrophotometrically).

### Example 1

### RCC Cell-Based Readout for Isolation of P53-Activating Agents

International Patent Application No. PCT/US2005/025884 (published as WO 2006/012419), the contents of which are described herein by reference, describes an RCC cell-based readout for screening compounds based on the ability to activate p53. Briefly, the transactivation function of p53 is inhibited in RCC cells by a previously unknown inhibitory factor, suggesting drug-mediated restoration of p53 function as an approach to selective killing of this type of tumor cells as well as other tumor cells with similar inhibition of p53. To test whether the reactivation of p53 would be toxic for RCC cells, we ectopically expressed p53 in five RCC-derived cell lines in an attempt to deplete the inhibitory factor. Cells were supplemented with integrated p53-responsive reporter (ConALacZ) to monitor p53 reactivation. p53 cDNA was transduced using a lentiviral vector with CMV promoter. p53-deficient lung adenocarcinoma cell line H1299, which are sensitive to wild type p53, and rat fibroblastoid cell line Rat1, which is resistant to human p53, were used as controls.

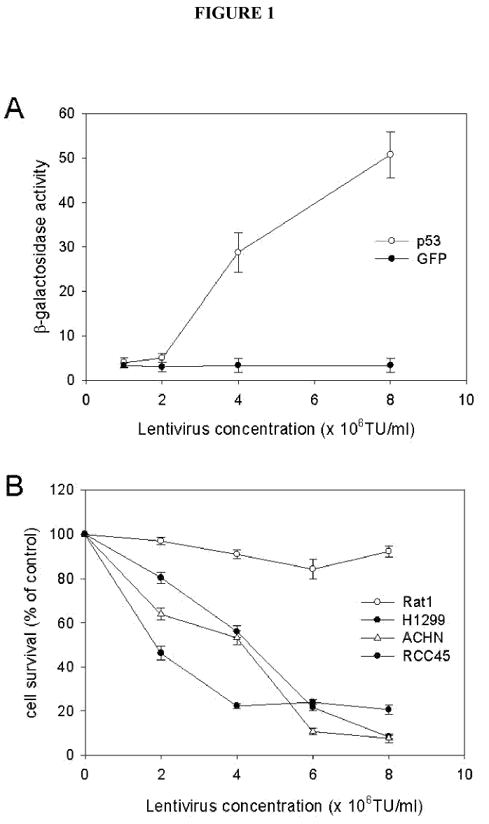

As shown in FIG. 1, dormant p53 in RCC may be reactivated, and that reactivation leads to tumor cell death. Starting from a certain level of expression, p53 became simultaneously cytotoxic and active in inducing the reporter in RCC45 cells (FIGS. 1a and b). This indicates that. cells, such as RCC cells, may be used in a cell-based reporter system to screen for agents that are capable of reactivating p53. This also indicates that reactivation of p53 in tumor cells, such as RCC cells, may be cytotoxic.

### Example 2

### Screening Chemical Library Identity Aminoacridines as a Potent P53 Activator in RCC

International Patent Application No. PCT/US2005/025884 (published as WO 2006/012419), the contents of which are described herein by reference, describes the screening and identification of p53 activating agents in the RCC cell-based readout. Briefly, we carried out a direct cell-based screening of chemicals capable of restoring p53 transactivation in RCC hoping to isolate small molecules with therapeutic potential that could also be used as tools for deciphering mechanisms of RCC specific p53 repression. RCC45ConALacZ cells were used to screen a diverse chemical library of 34,000 compounds (Chembridge Corporation). β-galactosidase activity was measured in cell lysates 24 hours after incubation with the compounds.

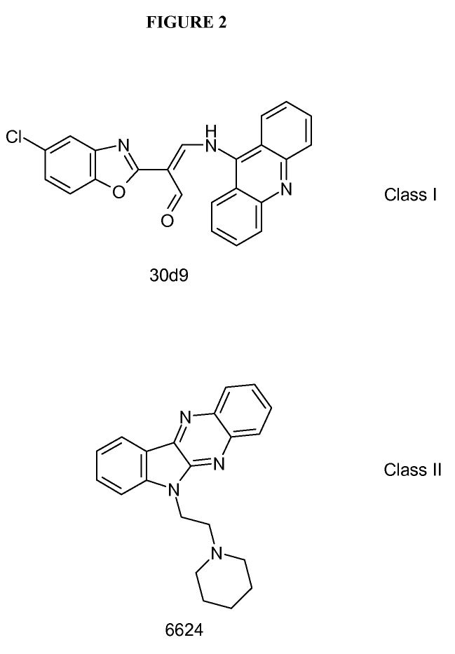

Twenty-eight compounds that induced β-galactosidase activity higher than that of 1 μM of doxorubicin were considered as primary hits. Three of the primary hits are shown in FIG. 2. The most active agent was compound 30d9, which caused a 22-fold induction of the reporter in RCC45 cells acting 7 times stronger than doxorubicin.

### Example 3

### Screening Focused Libraries

### Identification of Aminoacridines as a Potent P53 Activator in RCC

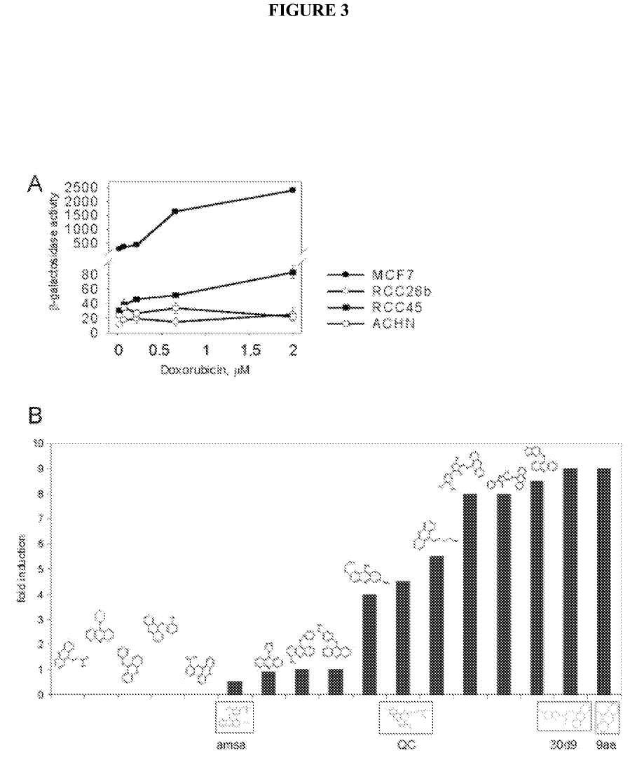

Focused libraries were synthesized and tested based on the primary hit of Class I (compound 30d9). FIG. 3a shows the p53 restoration activity of agents from the focused library based on compound 30d9, which is of the formula of compound 1. The library of structural analogues built around compound 30d9 and consisting of 40 chemicals was screened using the same cell-based reporter assay. Two agents of the formula of compound 1 were found to be active.

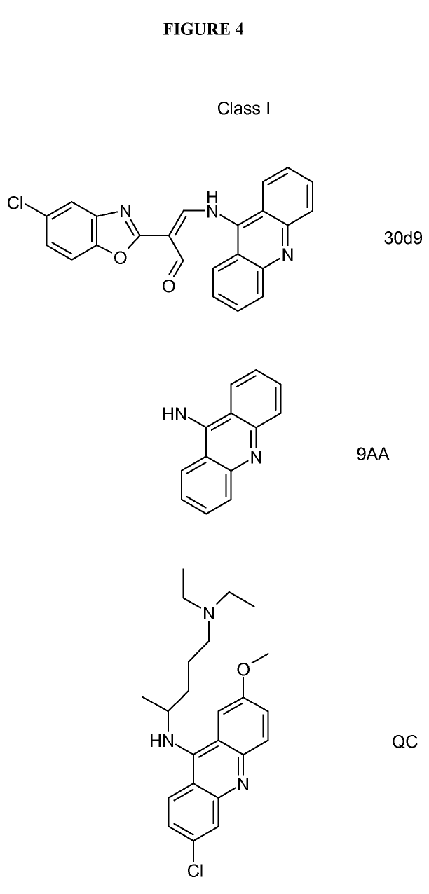

A library of 59 derivatives of compound 30d9 were then screened, including the anti-cancer agent amsacrine (amsa) and anti-malaria agent quinacrine. Twelve of the tested compounds reactivated p53 in RCC45 cells ranging in their activity similar to doxorubicin (e.g., amsa) to 7-10 folds stronger than doxorubicin, with compound 30d9 and 9AA being the strongest (FIG. 3B). Quinacrine showed an intermediate level of activity. SAR analysis indicated that Class I compounds capable of reactivating p53 are aminoacridines (FIG. 4).

### Example 4

### Screening Focused Libraries

### Identification of Ellipticine-Like Compounds as Potent P53 Activators in RCC

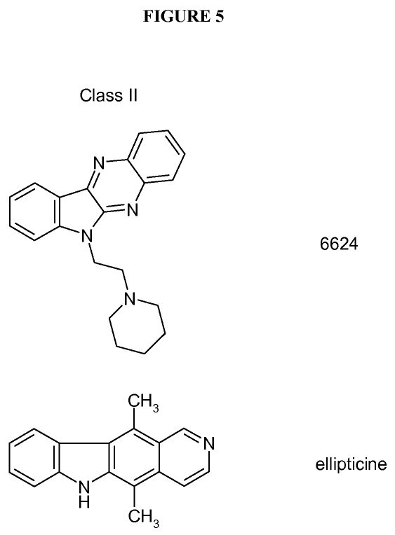

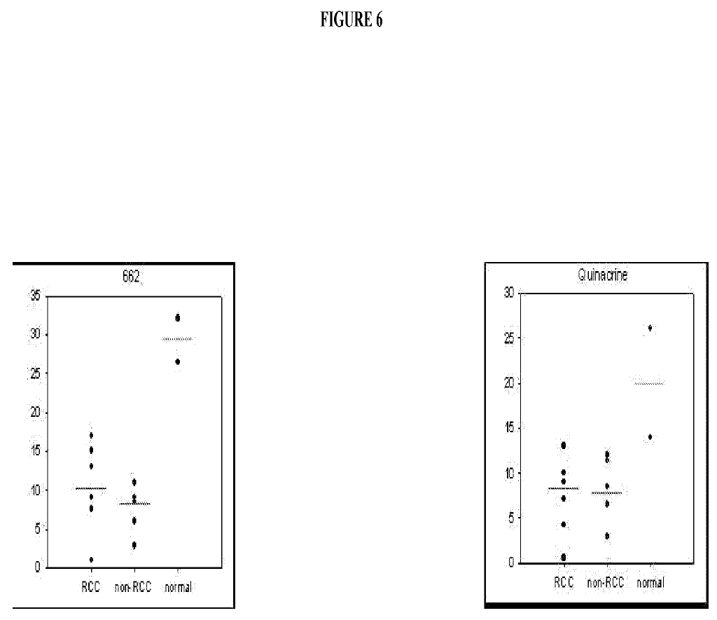

Focused libraries were also synthesized and tested based on the primary hit of Class II. The library of Class II structural analogues were screened using the same cell-based reporter assay. A number of compounds from the Class II focused library were identified (FIG. 5). One of the identified compounds was ellipticine.

### Example 5

### Additional Screening

Another library of 108 compounds was synthesized based on the similarity of hits from the Class I and Class II focused libraries. The library included compounds 6624, 6628 and 5219. The compounds were tested for p53 activation in HT1080-L cells and RCC45ConA-Luc cells.

Of the 108 compounds tested, 20 compounds induced p53 transactivation ≧2 fold. In order to rank the compounds, each compound was assigned a total score based on the criteria shown in Table 1.

TABLE 1

| Test | Cells | Criteria | | Score |
|---|---|---|---|---|
| fold - p53 activation | HT1080-L | >15 | fold | 3 |
| fold - p53 activation | HT1080-L | 10-15 | fold | 2 |
| fold - p53 activation | HT1080-L | <10 | fold | 1 |
| Emax - p53 activation | HT1080-L | 1-10 | μM | 3 |
| Emax - p53 activation | HT1080-L | 10-20 | μM | 2 |
| Emax - p53 activation | HT1080-L | >20 | μM | 1 |
| EC50 - p53 activation | HT1080-L | 0-10 | μM | 3 |
| EC50 - p53 activation | HT1080-L | 10-20 | μM | 2 |
| EC50 - p53 activation | HT1080-L | >20 | μM | 1 |
| fold - p53 activation | RCC45ConA-Luc | >5 | fold | 3 |
| fold - p53 activation | RCC45ConA-Luc | 2-5 | fold | 2 |
| fold - p53 activation | RCC45ConA-Luc | 0 | fold | 1 |
| Emax - p53 activation | RCC45ConA-Luc | 0-10 | μM | 3 |
| Emax - p53 activation | RCC45ConA-Luc | 10-20 | μM | 2 |
| Emax - p53 activation | RCC45ConA-Luc | >20 | μM | 1 |
| EC50 - p53 activation | RCC45ConA-Luc | 0-10 | μM | 3 |
| EC50 - p53 activation | RCC45ConA-Luc | 10-20 | μM | 2 |
| EC50 - p53 activation | RCC45ConA-Luc | >20 | μM | 1 |
| EC50 - inhibition of | SK-RC-45 | 0-10 | μM | 3 |
| NF-κB transactivation | | | | |
| EC50 - inhibition of | SK-RC-45 | 10-20 | μM | 2 |
| NF-κB transactivation | | | | |
| EC50 - inhibition of | SK-RC-45 | >20 | μM | 1 |
| NF-κB transactivation | | | | |
| EC50 - growth inhibit | NKE-TERT | 0-10 | μM | 3 |
| EC50 - growth inhibit | NKE-TERT | 10-20 | μM | 2 |
| EC50 - growth inhibit | NKE-TERT | >20 | μM | 1 |
| EC50 - growth inhibit | HT1080 sip53 | 0-10 | μM | 3 |
| EC50 - growth inhibit | HT1080 sip53 | 10-20 | μM | 2 |
| EC50 - growth inhibit | HT1080 sip53 | >20 | μM | 1 |
| EC50 - growth inhibit | HT1080 siGFP | 0-10 | μM | 3 |
| EC50 - growth inhibit | HT1080 siGFP | 10-20 | μM | 2 |
| EC50 - growth inhibit | HT1080 siGFP | >20 | μM | 1 |

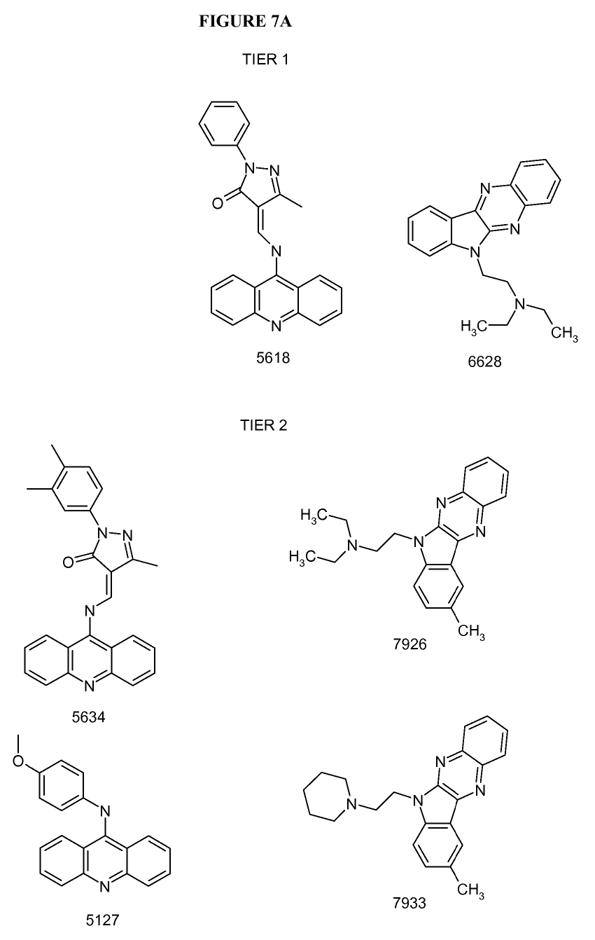

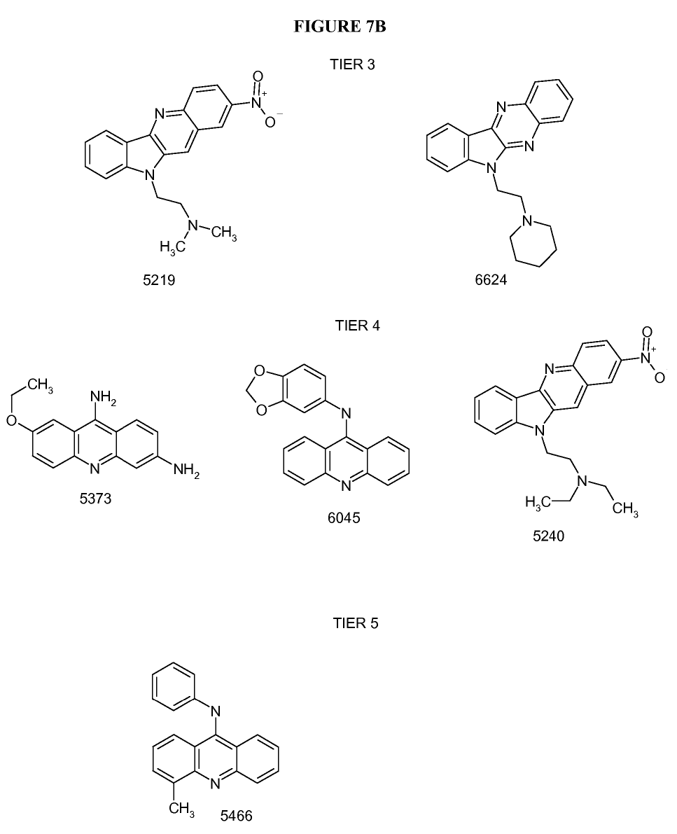

Based on the above criteria, the compounds (FIG. 7) were ranked in five Tiers, with Tier 1 having the highest cumulative score.

TABLE 2

| Tier | Compounds |
|---|---|
| 1 | 6628 and 5618, |
| 2 | 7926, 5634, 5127 and 7933 |
| 3 | 6624 and 5219 |
| 4 | 5373, 6045 and 5240 |
| 5 | 5466 |
| 6 | 5884, 6590, 7728, 7501, 7469 and 7693 |

The compounds of Tier 1 fall into two structural groups: 9aa-like and 6624-like. The compounds of Tier 2 also fall into two structural groups: 9aa-like and 6624-like. The compounds of Tier 3 are 6624-like. The compounds of Tier 4 fall into two structural categories: 9aa-like and 6624-like. The compounds of Tier 5 are 9aa-like.

1. A method of treating a condition associated with NF-κB activity comprising administering to a patient in need thereof a composition comprising an inhibitor of NF-κB.

2. The method of claim 1, wherein the NF-κB activity is constitutive or induced.

3. The method of claim 1, wherein the NF-κB activity is at a basal level.

4. The method of claim 1, wherein inhibition of NF-κB activates p53.

5. The method of claim 1, wherein the condition is cancer.

6. The method of claim 5, wherein the inhibition of NF-κB leads to activation of functionally impaired wild type p53.

7. The method of claim 5, wherein the cancer is selected from the group consisting of renal cell carcinoma, sarcoma, prostate cancer, breast cancer, pancreatic cancer, myeloma, myeloid and lymphoblastic leukemia, neuroblastoma, glioblastoma and a cancer caused by HTLV infection.

8. The method of claim 1, wherein the condition is inflammation, an autoimmune disease, graft versus host disease, or a condition associated with HIV infection.

9. The method of claim 1, wherein the condition is pre-cancerous cells which have acquired dependence on constitutively active NF-κB.

10. The method of claim 1, wherein the inhibitor of NF-κB is a compound of the formula: 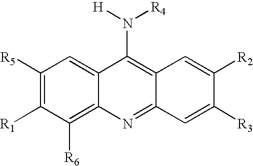
wherein,
R₁ is H or halogen;
R₂ is H or optionally substituted alkoxy group;
R₃ is H, optionally substituted alkoxy group or optionally substituted amino group;
R₄ is H, optionally substituted aliphatic group, optionally substituted aryl group, or optionally substituted heterocycle;
R₅ is H or optionally substituted alkoxy group;
R₆ is H or optionally substituted alkyl.

11. The method of claim 1, wherein the inhibitor of NF-κB is a compound selected from the group consisting of: 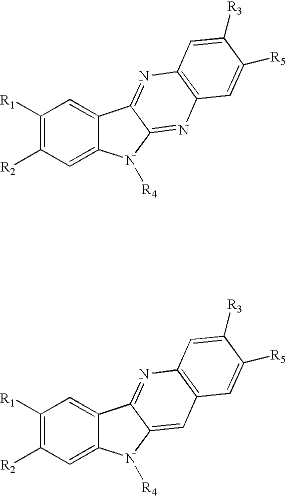
wherein,
R₁-R₃ and R₅ are individually H or optionally substituted alkoxy; and
R₄ is H or optionally substituted aliphatic, aryl, or heterocycle.

12. The method of claim 10, wherein the compound is set forth in FIGS. 2-5 and 7.

13. The method of claim 12, wherein the composition further comprises an activator of a death receptor of a TNF family polypeptide.

14. The method of claim 13, wherein the activator is a TNF family polypeptide selected from the group consisting of NGF, CD40L, CD137L/4-1BBL, TNF-α, CD134L/OX40L, CD27L/CD70, FasL/CD95, CD30L, TNF-β/LT-α, LT-β, and TRAIL.

15. A method of screening for an agent that activates functionally silent p53 comprising:
(a) adding a candidate agent to a cell comprising a p53-responsive reporter;
(b) measuring the level of signal of the p53-responsive reporter,
whereby an agent is identified by signal in (b) above a control.

16. The method of claim 15 wherein the cell comprises a functionally silent p53.

17. A method of screening for an agent that inhibits NF-κB comprising:
(a) adding a candidate agent to a cell comprising a p53-responsive reporter;
(b) measuring the level of signal of the p53-responsive reporter,
whereby an agent is identified by signal in (b) above a control.

18. The method of claim 17 wherein the cell comprises a functionally silent p53.

19. The method of claim 17 wherein the cell comprises an NF-κB transactivation complex.

20. The method of claim 11, wherein the compound is set forth in FIGS. 2-5 and 7.

21. The method of claim 20, wherein the composition further comprises an activator of a death receptor of a TNF family polypeptide.

22. The method of claim 21, wherein the activator is a TNF family polypeptide selected from the group consisting of NGF, CD40L, CD137L/4-1BBL, TNF-α, CD134L/OX40L, CD27L/CD70, FasL/CD95, CD30L, TNF-β/LT-α, LT-β, and TRAIL.
